(19) United States
(12) Patent Application Publication
(10) Pub. No.: US 2009/0069862 A1
(43) Pub. Date: Mar. 12, 2009

(54) Adaptive Telemetry Wakeup for an Implantable Medical Device

(76) Inventor: Brian Michael Shelton, Northridge, CA (US)

Correspondence Address: HENRICKS SLAVIN AND HOLMES LLP;SUITE 200, 840 APOLLO STREET, EL SEGUNDO, CA, 90245, US

(21) Appl. No.: 11/853,776

(22) Filed: Sep. 11, 2007

(51) Int. Cl. A61N 1/08

(52) U.S. Cl. 607/32

(57) ABSTRACT

A method for providing an adaptive telemetry wakeup for an implantable medical device includes accessing programmer usage statistics or patient controller usage statistics associated with an implantable medical device, and adjusting a wakeup protocol for the implantable medical device depending upon the programmer usage statistics or patient controller usage statistics.

### BACKGROUND OF THE INVENTIONS

1. Field of Inventions

The present inventions relate generally to implantable medical devices and providing telemetry wakeup for implantable medical devices.

2. Description of the Related Art

There are competing requirements for biomedical implant devices that are electronically powered. If such devices are rechargeable, there is the desire to maximize the recharge interval. If such devices are not rechargeable, there is the desire to consume as little power as possible so as to increase device longevity. However, it is also desirable to have a frequent wakeup period for the purpose of listening for communication with any possible ex-vivo monitoring or controlling devices.

The best way to satisfy both of these requirements would be to wake up only when the clinician (e.g., physician) or patient is about to communicate with the implant. However, this information is typically not known a priori.

It would be useful to be able to provide a telemetry wakeup for a implantable medical device that addresses the foregoing competing requirements.

### SUMMARY OF THE INVENTIONS

In an example embodiment, a method for providing an adaptive telemetry wakeup for an implantable medical device includes accessing programmer usage statistics associated with an implantable medical device, and adjusting a wakeup protocol for the implantable medical device depending upon the programmer usage statistics.

In an example embodiment, a method for providing an adaptive telemetry wakeup for an implantable medical device includes accessing patient controller usage statistics associated with an implantable medical device, and adjusting a wakeup protocol for the implantable medical device depending upon the patient controller usage statistics.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 illustrates an implantable medical device and example programmers/controllers with which the methods for providing an adaptive telemetry wakeup for an implantable medical device described herein can be employed.

FIG. 2 is a plan view of a patient controller in accordance with one embodiment of the present invention.

FIG. 3 is a block diagram of the patient controller of FIG. 2.

FIG. 4 is a plan view of a programmer in accordance with one embodiment of the present invention.

FIG. 5 is a plan view of a programmer in accordance with another embodiment of the present invention.

FIG. 6 is a flow chart in accordance with one embodiment of the present invention.

FIG. 6A conceptually illustrates processing of usage statistics over multiple days to determine an average (or median) pump read time.

FIG. 6B conceptually illustrates processing of usage statistics over multiple weeks to determine an average number of pump reads for each day of the week.

FIG. 7 is a flow chart in accordance with another embodiment of the present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

The present remote controls have application in a wide variety of medical device systems. One example of such a system is an implantable infusion device system and the present inventions are discussed in the context of implantable infusion device systems. The present inventions are not, however, limited to implantable infusion device systems and are instead also applicable to other medical device systems that currently exist, or are yet to be developed. For example, the present inventions are applicable to other ambulatory medical device systems. Such systems include, but are not limited to, externally carried infusion pump systems, implantable pacemaker and/or defibrillator systems, implantable neural stimulator systems, and implantable and/or externally carried physiologic sensor systems.

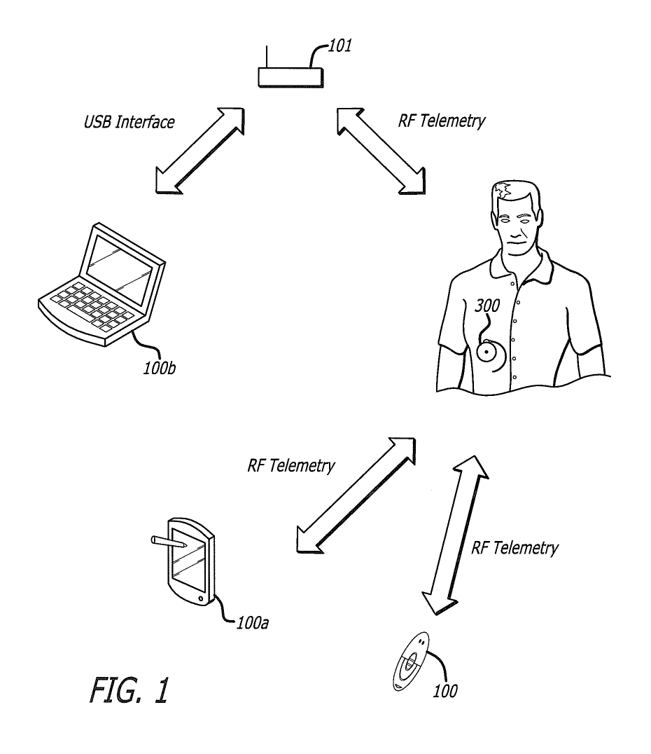

One example of an ambulatory medical device system in accordance with the present inventions is an implantable infusion device system. The implantable infusion device system may include any one of the remote controls described herein in combination with an implantable infusion device. FIG. 1 illustrates an implantable medical device 300 and example programmers/controllers with which the methods for providing an adaptive telemetry wakeup for an implantable medical device described herein can be employed. The example programmers/controllers includes a patient controller 100 (such as a remote control device), a programmer 100a (such as a portable computing device (PCD) or personal digital assistant (PDA)), and a clinician programmer 100b (such as a clinician programmer/field support system). In this example, the patient controller 100 and the programmer 100a include communication devices which facilitate radio frequency (RF) communications with the implantable medical device 300 so that RF telemetry can be communicated between the devices. Also in this example, the clinician programmer 100b is connected to a programmer interface module 101 with a USB Interface; the programmer interface module 101, in turn, facilitates RF communications between the clinician programmer 100b and the implantable medical device 300. It should be understood that other types of programmers/controllers as well as other communications interfaces can also be employed.

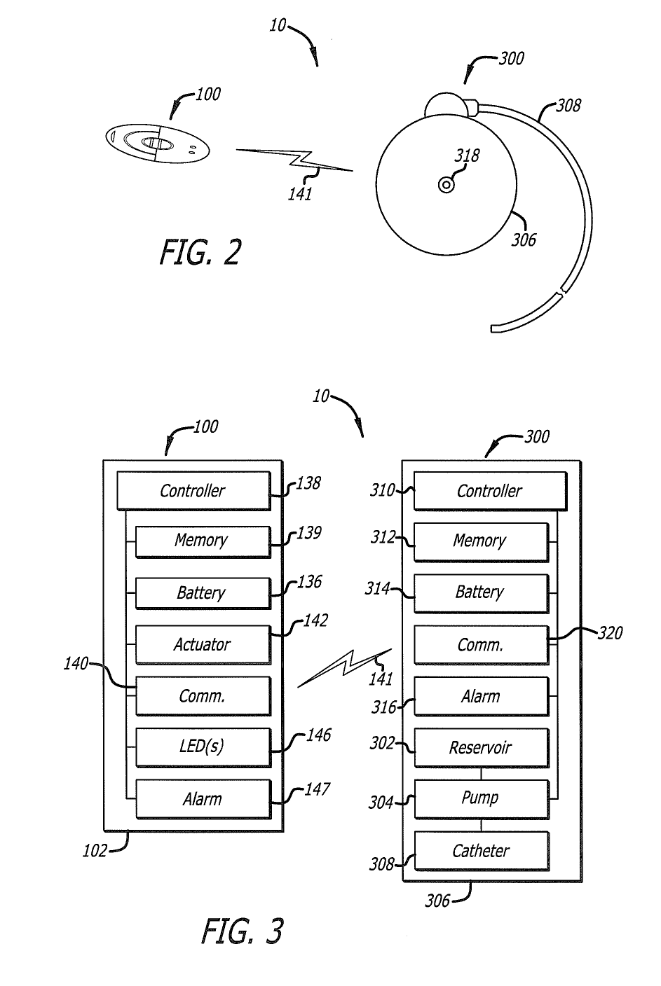

Referring to FIG. 2, in an example embodiment, an implantable medical device system 10 includes a patient controller 100 (in the form of a remote control) and an implantable medical device 300. In an example embodiment, the patient controller 100 includes a battery or other power source 136, a controller 138, such as a microprocessor, microcontroller or other control circuitry, memory 139, an actuator 142 with a movable element, and one or more LEDs 146 (and/or alarm 147). The memory 139 can also be contained within the controller 138 (e.g., within a microcontroller). By way of example and not of limitation, the alarm 147 can include one or more of an audio speaker and a vibration device. A communication device 140 (including an antenna if necessary) is also provided. The communication device 140 establishes a communications link 141, e.g., an RF communications link, with the implantable medical device 300. Although the present inventions are not limited to any particular communication device, in an example embodiment, the communication device 140 is a telemetry device that transmits an RF signal at a specified frequency or set of frequencies. In an example implementation, there are five channels. The RF signal may, in some instances, be a carrier signal that carries bit streams. The communication device 140 is also configured to receive signals from the implantable medical device 300. Other exemplary communication devices include oscillating magnetic field communication devices, static magnetic field communication devices, optical communication devices, ultrasound communication devices and direct electrical communication devices.

In this example embodiment, the implantable medical device 300 is an implantable infusion device and includes a medication reservoir 302 and a pump or other fluid transfer device 304 within a housing 306. The pump 304 transfers medication from the reservoir 302 through a catheter 308 to the target region within the body. Operation of the implantable medical device 300 is controlled by a controller 310, such as a microprocessor, microcontroller or other control circuitry, in accordance with instructions stored in memory 312. Power is provided by a battery or other power source 314. An alarm 316 (e.g., an audible alarm such as an audio speaker, and/or a vibration device) may also be provided in order to inform the patient, for example, when the amount of medication in the reservoir 302 is low or when the amount of energy stored in the battery 314 is low. A refill port 318, which allows the reservoir to be refilled while the implantable medical device 300 is within the patient, is positioned on the exterior of the housing 306.

A communication device 320 is also provided. In this example embodiment, the communication device 320 is configured to receive signals from, and transmit signals to, the patient controller 100. In an example embodiment, the communication device 320 is a telemetry device that transmits and receives RF signals at a specified frequency or set of frequencies. The RF (or other) signal may, in some instances, be a carrier signal that carries bit streams.

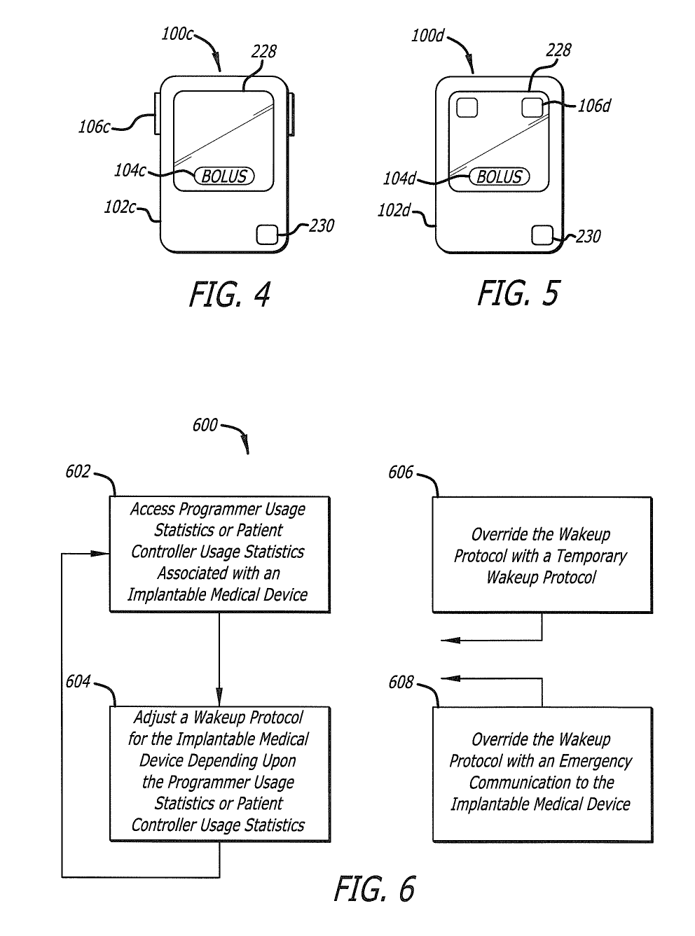

It should be noted here that, in the context of the present inventions, different types and/or combinations of user input devices can be employed with any given programmer/controller device. As illustrated for example in FIG. 4, the exemplary programmer 100c includes a housing 102c and a touch screen 228. A controller and a communication device (not shown) are also provided. The touch screen 228 may be used to display one or more button configurations in order to allow the user to accomplish various tasks. At least one of the displayed buttons is a bolus delivery button 104c. The housing 102c may also be provided with one or more button control elements 106c (e.g. buttons), which are operably connected to the controller, and a power on/off button 230.

One or more button control elements may, alternatively, be provided on a touch screen. Turning to FIG. 5, the exemplary programmer 100d includes a housing 102d, a touch screen 228 that may be used to, among other things, display a bolus delivery button 104d and a pair of button control elements 106d, and a power on/off button 230.

Methods described herein employ an adaptive approach to controlling telemetry wakeup for an implantable medical device. The energy requirements of a telemetry system are often a major limitation to the life of an implanted device. By reducing the “listening time” when the telemetry hardware of the device is powered according to the methods described herein, the longevity of the device can be extended. Moreover, by improving the telemetry scheme for establishing communication between a device controller/monitor and the implanted device, the methods described herein can reduce the surgical risks to the patient that are associated with more frequent device replacement.

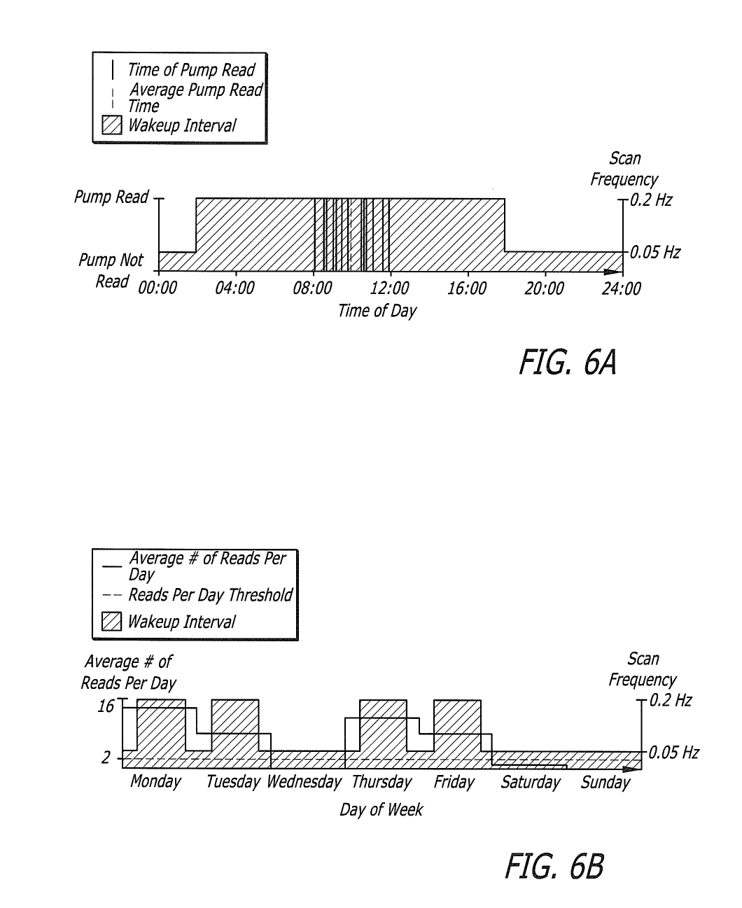

Referring to FIG. 6, in an example embodiment, a method 600 for providing an adaptive telemetry wakeup for an implantable medical device includes, at 602, accessing programmer usage statistics or patient controller usage statistics associated with an implantable medical device. For example, the programmer usage statistics or patient controller usage statistics are stored in a memory device that is accessible to the programmer or patient controller.

In an example embodiment, the programmer usage statistics are clinician-specific or clinic-specific. Thus, in such embodiments, data related to the actions of a particular clinician (e.g., physician or other medical professional) provide the programmer usage statistics.

In an example embodiment, the patient controller usage statistics are patient-specific. Thus, in such embodiments, data related to the actions of a particular patient provide the patient controller usage statistics.

Referring again to FIG. 6, the method 600 for providing an adaptive telemetry wakeup for an implantable medical device includes, at 604, adjusting a wakeup protocol for the implantable medical device depending upon the programmer usage statistics or patient controller usage statistics. Over time, as actions are taken by a programmer or patient with respect to usage of the implantable medical device, the programmer usage statistics or patient controller usage statistics change. As shown in FIG. 6, current (updated) programmer usage statistics or patient controller usage statistics are accessed by returning to 602. In this example embodiment, at 606, when a temporary wakeup protocol is initiated (e.g., by a clinician or patient), the wakeup protocol is overridden. Also, in this example embodiment, at 608, when an emergency communication to the implantable medical device is initiated, the wakeup protocol is overridden. Thus, the methods described herein facilitate receiving various interrupt-type commands which cause the adaptive wakeup protocol to be temporarily overridden.

There are various ways to adjust a wakeup protocol for the implantable medical device depending upon the programmer usage statistics or patient controller usage statistics. In an example embodiment, adjusting a wakeup protocol for the implantable medical device includes adjusting a length of time between telemetry wakeup intervals during which the implantable medical device listens for communications from a programmer device or patient controller device. In an example embodiment, adjusting a wakeup protocol for the implantable medical device includes adjusting the duration of a telemetry wakeup interval during which the implantable medical device listens for communications from a programmer device or patient controller device. The term “telemetry wakeup interval” means an interval of time during which the frequency of scanning for programmer or patient controller telemetry/data is elevated as compared to the scan frequency during sleep modes or other times outside the telemetry wakeup interval. For example, the scan frequency during a telemetry wakeup interval is 0.2 Hz, while the scan frequency during a sleep interval is 0.05 Hz.

In an example embodiment, adjusting a wakeup protocol for the implantable medical device includes partitioning the programmer usage statistics or patient controller usage statistics into groups corresponding to different portions of the week, and separately processing the groups of programmer usage statistics or patient controller usage statistics to adaptively adjust the wakeup protocol during each of the different portions of the week depending upon the programmer usage statistics or patient controller usage statistics associated with each of the different portions of the week. In an example embodiment, the different portions of the week include weekdays and the weekend. In an example embodiment, the different portions of the week include one or more clinic open intervals and clinic closed intervals. In an example embodiment, the different portions of the week include individual days. In an example embodiment, the different portions of the week include a portion identified by processing the programmer usage statistics or patient controller usage statistics. In an example embodiment, the different portions of the week include a portion adaptively identified by processing the programmer usage statistics or patient controller usage statistics.

In an example embodiment, adjusting a wakeup protocol for the implantable medical device includes applying a finite filter to the programmer usage statistics or patient controller usage statistics to select data to be processed (e.g., the “select data” includes data from a finite set bounded by a particular time interval, such as the last three months). In an example embodiment, adjusting a wakeup protocol for the implantable medical device includes adaptively adjusting the finite filter depending upon the programmer usage statistics or patient controller usage statistics (e.g., the time interval is modified depending upon the programmer usage statistics or patient controller usage statistics).

The finite filter can be adaptively adjusted depending upon day or week usage statistics. By way of example, and referring to FIG. 6A, an average (or median) pump read time can be determined by processing usage statistics for different numbers of days (e.g., 7 days, 14 days, etc.), and this in turn can be used to select an appropriate number of days over which it is deemed desirable to sample and average the pump read times, and/or to adjust the wakeup interval (e.g., to track the average or median pump read time, or to narrow or widen the wakeup interval). Also, by way of example, and referring to FIG. 6B, an average number of pump reads for each day of the week can be determined by processing usage statistics for different numbers of weeks (e.g., 2 weeks, 4 weeks, etc.), and this in turn can be used to select an appropriate number of weeks over which it is deemed desirable to sample and average the number of pump read per day, and/or to select an appropriate reads per day threshold, which can be used to facilitate automated selection of wakeup frequency during a particular wakeup interval (e.g., 0.2 Hz or 0.05 Hz) or to make other wake up frequency adjustments.

In an example embodiment, adjusting a wakeup protocol for the implantable medical device includes processing the programmer usage statistics to extract an interval of physician and/or clinician programmer usage. In another example embodiment, adjusting a wakeup protocol for the implantable medical device includes processing the patient controller usage statistics to extract an interval of patient controller usage.

In an example embodiment, adjusting a wakeup protocol for the implantable medical device includes processing the programmer usage statistics or patient controller usage statistics to find a mean of times (e.g., mean time-of-day) when the implantable medical device is linked to a programmer device or patient controller device.

In an example embodiment, adjusting a wakeup protocol for the implantable medical device includes processing the programmer usage statistics or patient controller usage statistics to identify minima in programmer usage or patient controller usage.

In an example embodiment, adjusting a wakeup protocol for the implantable medical device includes processing the programmer usage statistics or patient controller usage statistics to identify days which fall below a set programmer usage threshold or patient controller usage threshold.

In an example embodiment, adjusting a wakeup protocol for the implantable medical device includes processing a priori data relating to programmer usage or patient controller usage (e.g., advance knowledge of office hours, for embodiments where the implantable medical device keeps track of the time of day).

In an example embodiment, adjusting a wakeup protocol for the implantable medical device includes determining a day center. In an example embodiment, adjusting a wakeup protocol for the implantable medical device includes determining a day length. In an example embodiment, adjusting a wakeup protocol for the implantable medical device includes determining a list of active programming days.

Example methods described herein allow programmer usage statistics or patient controller usage statistics to be processed in order to provide a definition of a “day”, which is useful because there is no guarantee that the clock on the programmer or patient controller will be set correctly. Moreover, by analyzing programmer usage statistics or patient controller usage statistics on an on-going basis, changes in the usage activities or habits of the clinician or patient are adaptively taken into account when determining when the implantable medical device should listen, the duration of its listening intervals, etc.

In an example embodiment, a programmer (“Clinician Programmer”) that allows a physician or other clinician to query multiple implantable medical devices provides each of the implantable medical devices—when it talks with these devices—with a definition of a workday and a workweek based on the programmer usage statistics for all of the implantable medical devices.

For example, the statistical mean for the time of day when the Clinician Programmer communicates with the implantable medical devices can be used to set the center of the workday. By way of example, the length of the workday can either be fixed (e.g., 8 or 10 hours) or statistically determined.

In order to determine the beginning and the end of the workday, a simple minimum and maximum operation can be used. Alternately, the ratio of the number of outlying points to the total number of points can be examined.

By way of example, the workweek can be determined by either looking for the two days that had the fewest number of links, or by doing the same and then looking to verify that the percent of links on these days was lower than a threshold.

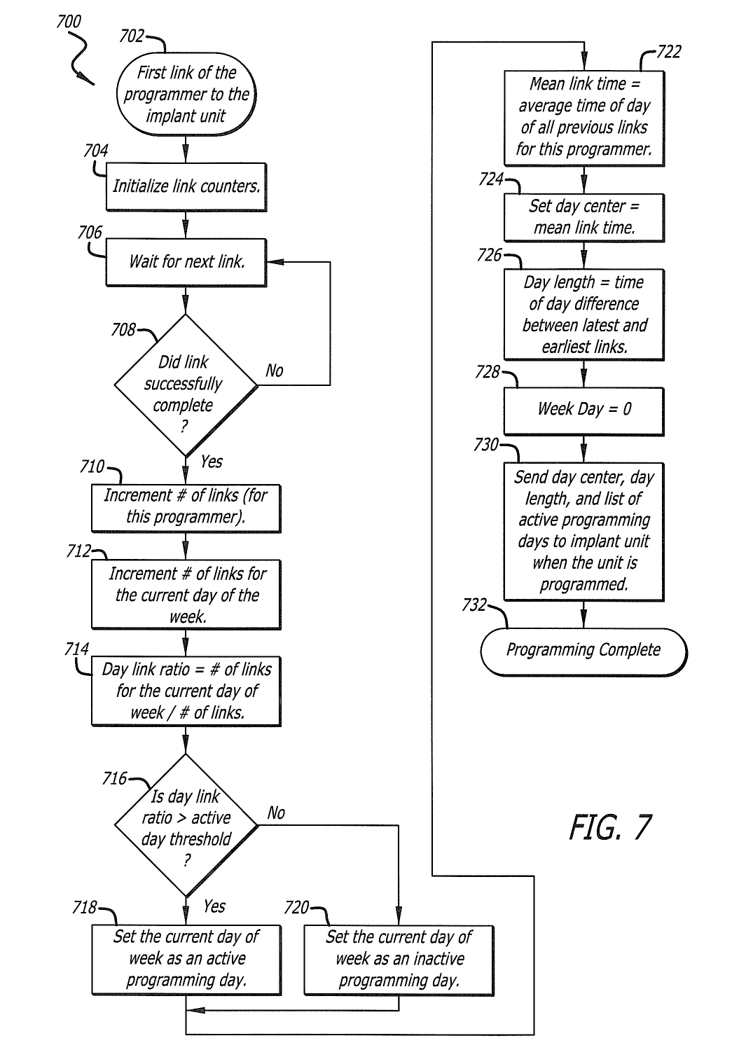

Referring to FIG. 7, in an example embodiment, a method 700 for providing an adaptive telemetry wakeup for an implantable medical device begins, at 702, when a first link of the programmer to the implant unit has been established. After initializing link counters, at 704, the program waits for the next link, at 706. At 708, a determination is made as to whether a link was successfully completed. If the determination is made in the negative, the program returns to wait for the next link, at 706. If a link was successfully completed, the “number of links” (for this programmer) is incremented, at 710, and the “number of links for the current day of the week” is incremented, at 712. Next, at 714, the “day link ratio” is determined as being equal to the number of links for the current day of the week divided by the number of links. At 716, a determination is made as to whether the day link ratio is greater than an active day threshold, i.e., a threshold set to indicate an active programming day. If the determination is made in the affirmative, the current day of the week is set as an active programming day, at 718. If the determination is made in the negative, the current day of the week is set as an inactive programming day, at 720. Next, at 722, the “mean link time” is determined as being equal to the average time of day of all previous links for this programmer. Alternately, a finite filter can be employed as discussed previously. At 724, the “day center” is set to be equal to the mean link time. At 726, the “day length” is set to be equal to the day difference between the latest and earliest links. At 730, day center, day length, and a list of active programming days are sent to the implant unit when the unit is programmed.

Although the inventions disclosed herein have been described in terms of the preferred embodiments above, numerous modifications and/or additions to the above-described preferred embodiments would be readily apparent to one skilled in the art. The inventions also include any combination of the elements from the various species and embodiments disclosed in the specification that are not already described. It is intended that the scope of the present inventions extend to all such modifications and/or additions and that the scope of the present inventions is limited solely by the claims set forth below.

What is claimed is:

1. A method for providing an adaptive telemetry wakeup for an implantable medical device, the method comprising:
accessing programmer usage statistics associated with an implantable medical device; and
adjusting a wakeup protocol for the implantable medical device depending upon the programmer usage statistics.

2. The method for providing an adaptive telemetry wakeup for an implantable medical device of claim 1, wherein the programmer usage statistics are clinician-specific.

3. The method for providing an adaptive telemetry wakeup for an implantable medical device of claim 1, wherein the programmer usage statistics are clinic-specific.

4. The method for providing an adaptive telemetry wakeup for an implantable medical device of claim 1, wherein adjusting a wakeup protocol for the implantable medical device includes adjusting a length of time between telemetry wakeup intervals during which the implantable medical device listens for communications from a programmer device.

5. The method for providing an adaptive telemetry wakeup for an implantable medical device of claim 1, wherein adjusting a wakeup protocol for the implantable medical device includes adjusting the duration of a telemetry wakeup interval during which the implantable medical device listens for communications from a programmer device.

6. The method for providing an adaptive telemetry wakeup for an implantable medical device of claim 1, wherein adjusting a wakeup protocol for the implantable medical device includes partitioning the programmer usage statistics into groups corresponding to different portions of the week, and separately processing the groups of programmer usage statistics to adaptively adjust the wakeup protocol during each of the different portions of the week depending upon the programmer usage statistics associated with each of the different portions of the week.

7. The method for providing an adaptive telemetry wakeup for an implantable medical device of claim 6, wherein the different portions of the week include weekdays and the weekend.

8. The method for providing an adaptive telemetry wakeup for an implantable medical device of claim 6, wherein the different portions of the week include one or more clinic open intervals and clinic closed intervals.

9. The method for providing an adaptive telemetry wakeup for an implantable medical device of claim 6, wherein the different portions of the week include individual days.

10. The method for providing an adaptive telemetry wakeup for an implantable medical device of claim 6, wherein the different portions of the week include a portion identified by processing the programmer usage statistics.

11. The method for providing an adaptive telemetry wakeup for an implantable medical device of claim 6, wherein the different portions of the week include a portion adaptively identified by processing the programmer usage statistics.

12. The method for providing an adaptive telemetry wakeup for an implantable medical device of claim 1, wherein adjusting a wakeup protocol for the implantable medical device includes applying a finite filter to the programmer usage statistics to select data to be processed.

13. The method for providing an adaptive telemetry wakeup for an implantable medical device of claim 12, wherein adjusting a wakeup protocol for the implantable medical device includes adaptively adjusting the finite filter depending upon the programmer usage statistics.

14. The method for providing an adaptive telemetry wakeup for an implantable medical device of claim 1, wherein adjusting a wakeup protocol for the implantable medical device includes processing the programmer usage statistics to extract an interval of physician and/or clinician programmer usage.

15. The method for providing an adaptive telemetry wakeup for an implantable medical device of claim 1, wherein adjusting a wakeup protocol for the implantable medical device includes processing the programmer usage statistics to find a mean time-of-day when the implantable medical device is linked to a programmer device.

16. The method for providing an adaptive telemetry wakeup for an implantable medical device of claim 1, wherein adjusting a wakeup protocol for the implantable medical device includes processing the programmer usage statistics to identify minima in programmer usage.

17. The method for providing an adaptive telemetry wakeup for an implantable medical device of claim 1, wherein adjusting a wakeup protocol for the implantable medical device includes processing the programmer usage statistics to identify days which fall below a set programmer usage threshold.

18. The method for providing an adaptive telemetry wakeup for an implantable medical device of claim 1, wherein adjusting a wakeup protocol for the implantable medical device includes processing a priori data relating to programmer usage.

19. The method for providing an adaptive telemetry wakeup for an implantable medical device of claim 1, wherein adjusting a wakeup protocol for the implantable medical device includes determining a day center.

20. The method for providing an adaptive telemetry wakeup for an implantable medical device of claim 1, wherein adjusting a wakeup protocol for the implantable medical device includes determining a day length.

21. The method for providing an adaptive telemetry wakeup for an implantable medical device of claim 1, wherein adjusting a wakeup protocol for the implantable medical device includes determining a list of active programming days.

22. The method for providing an adaptive telemetry wakeup for an implantable medical device of claim 1, further including the step of:
overriding the wakeup protocol with a temporary wakeup protocol.

23. The method for providing an adaptive telemetry wakeup for an implantable medical device of claim 1, further including the step of:
overriding the wakeup protocol with an emergency communication to the implantable medical device.

24. A method for providing an adaptive telemetry wakeup for an implantable medical device, the method comprising:
accessing patient controller usage statistics associated with an implantable medical device; and
adjusting a wakeup protocol for the implantable medical device depending upon the patient controller usage statistics.

25. The method for providing an adaptive telemetry wakeup for an implantable medical device of claim 24, wherein the patient controller usage statistics are patient-specific.

26. The method for providing an adaptive telemetry wakeup for an implantable medical device of claim 24, wherein adjusting a wakeup protocol for the implantable medical device includes adjusting a length of time between telemetry wakeup intervals during which the implantable medical device listens for communications from a patient controller device.

27. The method for providing an adaptive telemetry wakeup for an implantable medical device of claim 24, wherein adjusting a wakeup protocol for the implantable medical device includes adjusting the duration of a telemetry wakeup interval during which the implantable medical device listens for communications from a patient controller device.

28. The method for providing an adaptive telemetry wakeup for an implantable medical device of claim 24, wherein adjusting a wakeup protocol for the implantable medical device includes partitioning the patient controller usage statistics into groups corresponding to different portions of the week, and separately processing the groups of patient controller usage statistics to adaptively adjust the wakeup protocol during each of the different portions of the week depending upon the patient controller usage statistics associated with each of the different portions of the week.

29. The method for providing an adaptive telemetry wakeup for an implantable medical device of claim 28, wherein the different portions of the week include weekdays and the weekend.

30. The method for providing an adaptive telemetry wakeup for an implantable medical device of claim 28, wherein the different portions of the week include individual days.

31. The method for providing an adaptive telemetry wakeup for an implantable medical device of claim 28, wherein the different portions of the week include a portion identified by processing the patient controller usage statistics.

32. The method for providing an adaptive telemetry wakeup for an implantable medical device of claim 28, wherein the different portions of the week include a portion adaptively identified by processing the patient controller usage statistics.

33. The method for providing an adaptive telemetry wakeup for an implantable medical device of claim 24, wherein adjusting a wakeup protocol for the implantable medical device includes applying a finite filter to the patient controller usage statistics to select data to be processed.

34. The method for providing an adaptive telemetry wakeup for an implantable medical device of claim 33, wherein adjusting a wakeup protocol for the implantable medical device includes adaptively adjusting the finite filter depending upon the patient controller usage statistics.

35. The method for providing an adaptive telemetry wakeup for an implantable medical device of claim 24, wherein adjusting a wakeup protocol for the implantable medical device includes processing the patient controller usage statistics to extract an interval of patient controller usage.

36. The method for providing an adaptive telemetry wakeup for an implantable medical device of claim 24, wherein adjusting a wakeup protocol for the implantable medical device includes processing the patient controller usage statistics to find a mean of times when the implantable medical device is linked to a patient controller device.

37. The method for providing an adaptive telemetry wakeup for an implantable medical device of claim 24, wherein adjusting a wakeup protocol for the implantable medical device includes processing the patient controller usage statistics to identify minima in patient controller usage.

38. The method for providing an adaptive telemetry wakeup for an implantable medical device of claim 24, wherein adjusting a wakeup protocol for the implantable medical device includes processing the patient controller usage statistics to identify days which fall below a set patient controller usage threshold.

39. The method for providing an adaptive telemetry wakeup for an implantable medical device of claim 24, wherein adjusting a wakeup protocol for the implantable medical device includes processing a priori data relating to patient controller usage.

40. The method for providing an adaptive telemetry wakeup for an implantable medical device of claim 24, wherein adjusting a wakeup protocol for the implantable medical device includes determining a day center.

41. The method for providing an adaptive telemetry wakeup for an implantable medical device of claim 24, wherein adjusting a wakeup protocol for the implantable medical device includes determining a day length.

42. The method for providing an adaptive telemetry wakeup for an implantable medical device of claim 24, wherein adjusting a wakeup protocol for the implantable medical device includes determining a list of active programming days.

43. The method for providing an adaptive telemetry wakeup for an implantable medical device of claim 24, further including the step of:
overriding the wakeup protocol with a temporary wakeup protocol.

44. The method for providing an adaptive telemetry wakeup for an implantable medical device of claim 24, further including the step of:
overriding the wakeup protocol with an emergency communication to the implantable medical device.
